(19) United States
(12) Patent Application Publication
(10) Pub. No.: US 2015/0289785 A1
(43) Pub. Date: Oct. 15, 2015

(54) Apparatus and Method for Monitoring Respiration Volumes and Synchronization of Triggering in Mechanical Ventilation by Measuring the Local Curvature of the Torso Surface

(71) Applicant: DIASENS D.O.O., Belgrade (RS)

(72) Inventors: Bosko BOJOVIC, Belgrade (RS); Miodrag VUKCEVIC, Belgrade (RS); Jovana PETROVIC, Belgrade (RS); Marija PETROVIC, Belgrade (RS); Igor ILIC, Belgrade (RS); Aleksandar DANICIC, Belgrade (RS); Thomas ALLSOP, North Lincolnshire (GB); Ljupco HADZIEVSKI, Belgrade (RS)

(21) Appl. No.: 14/385,458

(22) PCT Filed: Aug. 29, 2013
(86) PCT No.: PCT/RS2013/000016

§ 371 Date: Sep. 15, 2014

(30) Foreign Application Priority Data
Aug. 30, 2012 (RS) P-2012/0373

(51) Int. Cl. A61B 5/113; A61B 5/0402; A61B 5/0205; A61M 16/00; A61B 5/00

(52) U.S. Cl. CPC A61B 5/1135; A61M 16/0069; A61B 5/7203; A61B 5/0205; A61B 5/0402; A61B 5/091

(57) ABSTRACT

The invention is related to a device and method for monitoring respiration, movements in mechanical ventilation in order to provide a non-pneumatic triggering variable for achieving patient-ventilator asynchrony and continuous measurement of tidal volumes. The method is based on measuring the curvature of the patient's torso surface using a single LPG (Long Period Grating) fiber-optic sensor attached to a surface of the torso in an area having high stiffness of the underlying tissue, such as the area of the lower ribs close to the sternum.

### TECHNICAL FIELD

The present invention relates to a sensor for measuring the respiration volume by means of measuring thoracic movements, specifically the local curvature variation of the surface of the human torso.

### BACKGROUND ART

This invention relates to a method and apparatus for continuous monitoring of the respiration of patients, particularly critically ill patients in intensive care units. In medicine, mechanical ventilation is a method to mechanically assist or replace spontaneous breathing. This may involve a machine called ventilator. There are two main types of mechanical ventilation: 1) invasive ventilation, using tracheal intubation (a tube is inserted through the nose or mouth and advanced into the trachea) and 2) non-invasive ventilation, using an oronasal mask or a mouthpiece. In mechanical ventilation, measurement (or at least an estimate) of the tidal volume (Vt, the volume of air moved into or out of the lungs during quiet breathing) is necessary to ensure adequate ventilation. In non-invasive ventilation, measurement can be affected by air leaks, which is an important problem commonly occurring when oronasal masks are used. A method of direct monitoring of the lung volume of patients, independent of air leaks, would enable virtually error-free measurement of the tidal volume.

From the early 1980s, a new generation of ventilators capable of providing assisted mechanical ventilation has been developed. These ventilators are equipped with a pneumatic sensor designed to detect the start of patient's inspiratory effort. The sensor triggering mechanism detects inspiration by means of a pneumatic signal generated by the patient's inspiratory effort and measured in the ventilatory circuit, i.e., pressure, flow or volume. In response, the ventilator will assist the patient's inspiratory effort.

Patient-ventilator asynchrony is referred to as the uncoupling of the mechanically delivered breath (ventilator) and neural respiratory effort (patient). This asynchrony imposes an additional burden on the respiratory system and may increase the morbidity of critically ill patients. Patient-ventilator asynchrony is present in 25% of mechanically ventilated patients in the intensive care units. Patient ventilator asynchrony during the triggering process appears in the following forms: autotriggering (triggering in the absence of inspiratory muscle contraction), excessive triggering delay (delay between the beginning of the inspiratory effort and ventilator triggering) and ineffective efforts (the inability of the patient inspiratory effort to trigger the ventilator). All pneumatic-triggering variables may be affected by air leaks. Theoretically, time delay between the neural activity and the initiation of inspiration is about 20 ms (Verbrugghe W, Jorens P G: Neurally adjusted ventilatory assist: a ventilation tool or a ventilation toy?, Respir Care. 2011 March; 56(3):327-35). When applied to lung simulators, mechanical ventilators with pneumatic triggering work with triggering delays as low as 50 ms (http://www.hamilton-medical.ch/clinical-resources/white-papers.html), but applied to patients the triggering delays reach 250-550 ms (Spahija J et al., Patient-ventilator interaction during pressure support ventilation and neurally adjusted ventilatory assist. Crit Care Med. 2010 February; 38(2):518-26)

A method of directly monitoring the lung volume of patients would provide a non-pneumatic triggering variable independent on air leaks.

The measurement of EELV (end-expiratory lung volume) is important for ventilator settings in patients with acute lung injury (ALI) and chronic obstructive pulmonary disease (COPD). EELV is measured compared to FRC (Functional Residual Capacity). EELV is defined as the lung volume at the end of expiration during the ventilator-assisted breathing at different levels of Positive end-expiratory pressure (PEEP) applied by the ventilator, while FRC is the volume of air present in the lungs at the end of passive expiration during normal breathing (without ventilator). While the measurement of Vt comprises relative measurement of lung volume, i.e. difference between minimal and maximal volumes in each breath, the measurement of EELV is dependent on absolute lung volume; thus, Unlike the Vt, the EELV monitoring is significantly influenced by the baseline drift during the lung-volume measurement in mechanical ventilation.

### Background Technology in Monitoring Lung Volumes

There are two non-pneumatic methods for measuring lung volumes: Respiratory Inductance Plethysmography (RIP) and Electrical impedance tomography (EIT).

Respiratory Inductance Plethysmography (RIP) is a noninvasive method for determination of changes in thoracic volume. It is used to monitor tidal volume (Vt) and to detect changes in end-expiratory lung volume (EELV). The method is based on the work of Konno et. al (Konno K, Mead J., Measurement of the separate volume changes of rib cage and abdomen during breathing. J Appl Physiol. 1967 March; 22(3):407-22.) that demonstrates that movements of the respiratory system can be approximated with the sum of the volume changes of the rib cage and abdominal compartments.

Respiratory inductive plethysmographs are generally composed of two elastic bands that are placed around the rib cage and abdomen for the movement monitoring. Two insulated sinusoid wire coils are placed within elastic and adhesive transducer bands. The bands are placed around the rib cage under the armpits and around the abdomen at the level of the umbilicus. They are connected to an oscillator and subsequent frequency demodulation electronics to obtain digital waveforms at the output. During inspiration the cross-section of the rib cage and abdomen increases altering the self-inductance of the coils. Thus, the electrical signals are proportional to the movements of the thoracic and abdominal compartments, each band producing an independent signal. Therefore, the sum of these two signals has to be calibrated against a known gas volume, using a simultaneous recording of the breathing volume by a spirometer or pneumotachometer (PNT).

The procedure of RIP calibration, which is considered the gold standard (Barbosa R C et al., Respiratory inductive plethysmography: a comparative study between isovolume maneuver calibration and qualitative diagnostic calibration in healthy volunteers assessed in different positions. J Bras Pneumol. 2012 April; 38(2):194-201.), comprises an isovolumetric maneuver performed by the patient several times. During the isovolumetric maneuver the subject is voluntary shifting the volumes back and forth between the rib cage and abdomen while the airway openings are occluded. Untrained subjects, however, often find it difficult to perform the isovolume maneuver.

Another calibration method often used that does not require this special breathing maneuver is QDC—Qualitative Diagnostic Calibration. QDC is carried out during a 5 min period of natural breathing, and requires that a subject maintains the same breathing pattern during the whole measurement. QDC provides the correct calibration factor only when applied to a set of breaths with constant or quasi-constant tidal volumes (De Groote et al., Mathematical assessment of qualitative diagnostic calibration for respiratory inductive plethysmography, J Appl Physiol 90:1025-1030, 2001). For this and similar reasons, although more comfortable for patients than the one using the isovolumetric maneuver, the accuracy of QDC calibration is often questioned. Whereas some authors report a good accuracy of measuring Vt using RIP (Valta P et al., Evaluation of Respiratory Inductive Plethysmography in the Measurement of Breathing Pattern and PEEP-Induced Changes in Lung Volume, Chest 1992; 102; 234-238; Blankman P. et al., Lung monitoring at the bedside in mechanically ventilated patients, Curr Opin Crit Care 2012, 18:261-266), others report accuracy that is “poor” (Strömberg N., Error analysis of a natural breathing calibration method for respiratory inductive plethysmography, Med Biol Eng Comput. 2001 May; 39(3):310-4; Whyte K F, Accuracy of respiratory inductive plethysmograph in measuring tidal volume during sleep. J Appl Physiol. 1991 November; 71(5):1866-71.), “not sufficiently accurate for clinical use” (Werchowski J L, Inductance plethysmography measurement of CPAP-induced changes in end-expiratory lung volume. J Appl Physiol. 1990 April; 68(4):1732-8.) or “not consistently precise enough for quantitative measurements of Vt in mechanically ventilated patients” (Neumann P, Evaluation of Respiratory Inductive Plethysmography in Controlled Ventilation, Chest 1998; 113; 443-451).

The RIP method suffers from a large baseline drift that jeopardizes accurate EELV determination (Neumann P, Evaluation of Respiratory Inductive Plethysmography in Controlled Ventilation, Chest 1998; 113; 443-451) and, consequently, has been largely abandoned (Grivans C. et al., Positive end-expiratory pressure-induced changes in end-expiratory lung volume measured by spirometry and electric impedance tomography Acta Anaesthesiol Scand 2011; 55: 1068-1077). For these and other reasons, RIP is rarely used in optimizing ventilator settings (Blankman P. et al., Lung monitoring at the bedside in mechanically ventilated patients, Curr Opin Crit Care 2012, 18:261-266).

RIP is widely used in diagnostics of Obstructive Sleep Apnea (OSA), as a part of Polysomnographic devices (Masa J. et al., Alternative Methods of Titrating Continuous Positive Airway Pressure, Am J Respir Crit Care Med Vol 170. pp 1218-1224, 2004). A practical problem encountered in inductive plethysmography is that the calibration coefficients depend on the position of the bands around thorax and abdomen. Consequently, the application of inductive plethysmography during sleep includes risk of providing inaccurate volume values, due to the displacement of the bands due to patient movement during sleep. (Farre R. et al., Noninvasive monitoring of respiratory mechanics during sleep, Eur Respir J 2004; 24: 1052-1060). Thus, RIP may produce poor correlation with a direct measurement of tidal volume by pneumotachograph during sleep (Whyte K F et al., Accuracy of respiratory inductive plethysmograph in measuring tidal volume during sleep. J Appl Physiol. 1991 November;71(5):1866-71.), and a number of apneas may not be detected (Weese-Mayer D., Comparison of Apnea Identified by Respiratory Inductance Plethysmography with That Detected by End-tidal CO2 or Thermistor Am J Respir Crit Care Med Vol 162. pp 471-480, 2000).

Electrical impedance tomography (EIT) is a technique based on high-frequency low-amplitude electrical currents injection and voltage measurements using electrodes on the skin surface (typically 16 or 32 electrodes) in one cross-section of the thorax—the EIT eclipse (usually just above the diaphragm), generating cross-sectional images that represent impedance change in the corresponding slice of the thorax. EIT may be used as a bedside method that allows for noninvasive measurements of changes in the regional lung parameters, such as regional ventilation and alveolar recruitment (opening of closed alveoli).

There have been attempts to monitor global lung volume changes using EIT. The relative change in electrical conductivity for the entire torso was estimated by the sum of all pixels in an EIT image. Although EIT has been shown to be very precise and reproducible when looking at regional ventilation, this does not hold for the total lung volume (Meier T. et al., Assessment of regional lung recruitment and derecruitment during a PEEP trial based on electrical impedance tomography. Intensive Care Med. 2008 March; 34(3):543-50. Epub 2007 Jul. 25). The change in the sum of all pixels in an EIT image reflects impedance variations in one cross-section of the thorax, while lung volume is a global parameter of the whole lungs (Hinz J. et al., End-expiratory lung impedance change enables bedside monitoring of end-expiratory lung volume change, Intensive Care Med (2003) 29:37-43). Also, at increasing lung volume the lung regions move along the cranio-caudal axis, and hence the individual pixels of the EIT map may no longer correspond to the same lung regions (Schibler A, Calzia E, Electrical impedance tomography: a future item on the “Christmas Wish List” of the intensivist? Intensive Care Med (2008) 34:400-401). Furthermore, since the diaphragm moves during the lung volume changes, the results can be influenced by the diaphragm's entering the EIT eclipse (Hinz J. et al., End-expiratory lung impedance change enables bedside monitoring of end-expiratory lung volume change, Intensive Care Med (2003) 29:37-43). It was found that the assumption of a strictly linear relation between the total lung volume and the EIT impedance change cannot be used to calculate EELV (Bikker I. et al., Lung volume calculated from electrical impedance tomography in ICU patients at different PEEP levels, Intensive Care Med (2009) 35:1362-1367).

### Background Technology in Achieving Patient-Ventilator Synchrony

Conventional methods for achieving patient-ventilator synchrony use pneumatic triggering variables. Pressure, flow, or volume signals are used to detect patient's respiratory effort in order to trigger a breath delivery by a mechanical ventilator.

Neurally Adjusted Ventilatory Assist (NAVA) is a technology used in mechanical ventilation. NAVA delivers ventilation assist in proportion to and in synchrony with patient's respiratory efforts, as reflected by Edi signal. This signal represents the electrical activity of the diaphragm, the body's principal breathing muscle. NAVA captures the electrical activity of the diaphragm (Edi) invasively, by using a special gastric tube (Edi catheter) placed into the patient's esophagus. This signal is fed to the ventilator and used to assist the patient's breathing in synchrony with and in proportion to the patient's own breathing efforts. Since NAVA is triggered by a signal from the patient's diaphragm, it is not dependent on a pneumatic airway signal and should theoretically have shorter trigger delay and reduced response time of the ventilator compared to the pneumatic trigger (Clement K. et al., Neurally triggered breaths reduce trigger delay and improve ventilator response times in ventilated infants with bronchiolitis, Intensive Care Med (2011) 37:1826-1832). In contrast to the conventional pneumatically-driven modes, NAVA has been shown to improve patient-ventilator interaction and yield a remarkable reduction in any asynchronies (Navalesi P. et al.: Chapter 8. Neurally adjusted ventilatory assist, in: New Developments in Mechanical Ventilation Edited by M. Ferrer and P. Pelosi. European Respiratory Society Monographs, Vol. 55. 2012. P. 116-123).

Patient-ventilator asynchrony is present in 25% of mechanically ventilated patients in the intensive care unit and may contribute to patient discomfort, higher use of sedation, development of delirium, ventilator-induced lung injury, prolonged mechanical ventilation, and ultimately mortality (Verbrugghe W, Jorens P G: Neurally adjusted ventilatory assist: a ventilation tool or a ventilation toy?, Respir Care. 2011 March; 56(3):327-35).

The main drawback of NAVA technology is its invasiveness. It uses a catheter placed into the patient's esophagus and requires draining gastric content via the nasogastric tube prior to catheter placement, etc., thus making the ventilation treatment more complicated and increasing risk and patient discomfort.

During NAVA, correct placement of the Edi-catheter is mandatory to deduce a reliable Edi signal for respirator control. The position of the diaphragm depends on the application of Positive End-Expiratory Pressure (PEEP), body position and intra-abdominal pressure (IAP) (Barwing J. et al., Evaluation of the catheter positioning for neurally adjusted ventilatory assist, Intensive Care Med (2009) 35:1809-1814). One method to predict the correct position of a gastric feeding tube is based on the measurement of the distance from the nose to the ear lobe and then to the xiphoid process of the sternum—the NEX distance (also proposed by the NAVA system manufacturer—Maquet Critical Care, Solna, Sweden). Edi signal obtained by the NEX distance method is suitable for running NAVA in about two thirds of patients—72% (Barwing J. et al., Evaluation of the catheter positioning for neurally adjusted ventilatory assist, Intensive Care Med (2009) 35:1809-1814). In remaining patients, a catheter positioning procedure monitored by a special tool implemented in the ventilator needs to be applied. In this procedure, the method to determine the optimal position of the Edi catheter is based on the quality and amplitude of the Edi signal and the trans-esophageal electrocardiogram (TECG). The catheter is inserted nasally to the maximum distance of 80 cm and pulled out in steps of 1 cm, while recording Edi and ECG signals for a 60 s period in each step. In some cases the suspected malposition needs to be verified by a chest X-ray (Barwing J. et al., Influence of body position, PEEP and intraabdominal pressure on the catheter positioning for neurally adjusted ventilatory assist, Intensive Care Med (2011) 37:2041-2045). This procedure may be time consuming, which is another potential drawback of the NAVA method.

### Other Technologies for Monitoring Lung Volumes

There are other technologies for monitoring lung volume that are mainly used in laboratory respiration studies, but have not made significant clinical impact:

Fiber-optic respiratory plethysmography (FORP) is a technical modification of RIP technology. The device incorporates the idea of using thoracic and abdominal belts, like in the conventional inductance plethysmography, to determine dynamic changes in the thoracic and abdominal wall circumference, but uses an optical fiber woven into the belts rather than the usual wire coils (Davis C. et al., A new fibre optic sensor for respiratory monitoring. Australas Phys Eng Sci Med. 1997 December; 20(4):214-9).

Optoelectronic Plethysmography (OEP) is a method to evaluate ventilation through an external measurement of the chest wall surface motion. A number of small reflective markers are placed on the thoraco-abdominal surface by adhesive tapes. A system of four television cameras connected to an automated motion analyzer measures three-dimensional coordinates of these markers and the enclosed volume is computed by connecting the points to form triangles (Aliverti A. et al., Optoelectronic Plethysmography in Intensive Care Patients, Am J Respir Crit Care Med Vol 161. pp 1546-1552, 2000). The method is used in laboratory respiration studies.

Respiratory Movement Measuring Instrument (RMMI) is a method similar to the Optoelectronic Plethysmography (OEP). It uses 6 ultrasound sensors mounted on a rigid frame to detect motion of 6 reflective markers (“landmarks”) placed on the thoraco-abdominal surface by adhesive tapes (Ragnarsdóttir M. Breathing Movements and Breathing Patterns among Healthy Men and Women 20-69 Years of Age, Respiration 2006; 73:48-54). The method is used in laboratory respiration studies.

Plethysmography based on LPG sensors (PLPG). This method is based on a series of fiber optic curvature sensors on a garment that are used to monitor thoracic and abdominal movements during respiration (Allsop T. et al., Application of long-period-grating sensors to respiratory Plethysmography, J Biomed Opt. 2007 November-December; 12(6):064003). The fiber optic curvature sensors are based on long-period gratings (LPG sensors). Each sensor consists of a fiber long-period grating laid on a carbon fiber ribbon and encapsulated in a low-temperature curing silicone rubber. An array of nine curvature sensors is placed in a series of pockets on a Lycra vest. The electronic/optical interrogation device monitors changes in attenuation bands of the LPG transmission spectra induced by bending.

This promising technology is aimed at using the curvature data to reconstruct the shape of thorax and abdomen, allowing absolute volumetric data to be obtained without any calibration. An attempt was also made to apply a sensing array simply calibrated using linear regression. The sensing array was used to record curvature changes during inspiration and expiration; the flow being simultaneously recorded at the mouth of the subject by a spirometer. Linear regression was applied to find the relationship between the respiratory volumes measured by spirometer and the measured curvatures, which can serve to predict tidal volumes from the curvature measurement. The volumetric error of 6-12% was found.

Along with the promising results, some drawbacks of the technique were reported (Allsop T. et al., Application of long-period-grating sensors to respiratory Plethysmography, J Biomed Opt. 2007 November-December; 12(6):064003): a) there is an overall constant volume error observed between successive measurements, so that the absolute measured volume cannot be inferred from a simple linear combination of the outputs, probably due to the movement of the vest between successive measurements, and b) sharp bending or buckling of the sensors may cause a nonuniform curvature, which could significantly distort the Shape of the LPGs' attenuation bands, leading to unreliable results.

### SUMMARY OF INVENTION

The shortcomings of the prior art measurements of respiration explained above can be overcome by the reconstruction of respiratory volumes from the change of the human torso curvature during breathing. This approach uses only one LPG curvature sensor placed to the highly stiff area of the torso, which is that where the underlying tissues are bone or cartilage. Such an area is the lower rib cage, preferably around the ribs 6-8, between two lines parallel to the sternum and located at about 10 cm to the left and to the right from the sternum.

It was found that when a human subject is in the supine position, the change in the torso curvature in this area is linearly proportional to the change in lung volume, that the measured points have small scattering around the line, that the measured volume agrees excellently with the reference measurement and that the linear dependence is maintained over long periods of breathing. To obtain the calibration curve, one needs to measure breathing volume over a short period of breathing by some direct method, like spirometer or pneumotachometer, while simultaneously measuring the curvature. Fast response and high sensitivity of LPG sensors enable activation signal generation with a minimal delay.

### Fibre LPG (Long Period Grating) Curvature Sensor

In the preferred embodiment, the measurement of curvature is based on long-period grating fibre-optical sensor. The long-period grating is a device that consists of a periodic change in the refractive index or the fibre geometry along the fibre length with the typical period of several hundred micrometers. The LPG couples core modes with the resonant co-propagational radiation cladding modes, which results in attenuation bands in the transmission spectrum (T. Erdogan, Cladding-mode resonances in short- and long-period fibre grating filters, Journal of Optical Society of America A 14(8) 1760-1773, 1997). Sensitivity of the LPG to a change in the grating curvature is due to a change in the effective propagation constants and mode profiles of the resonant modes, which is the consequence of the refractive index change across the fibre caused by bending. In general, the band central wavelengths and magnitudes of attenuation bands are sensitive to the forces applied to the fibre (pressure, bending) and environmental conditions (temperature, surrounding refractive index).

Curvature sensor consists of a fibre into which a long-period grating is inscribed. The fibre with the grating is then encapsulated in a silicone rubber to achieve isolation from temperature fluctuations. The fibre with the sensor is connected to an optoelectronic device that converts the curvature signal into an analogue electric signal.

The first step of the measurement procedure is calibration. It comprises simultaneous measurement of short breathing periods by a curvature sensor and a pneumotachometer or spirometer, the application of which requires using either an oronasal mask or tube with a mouthpiece. The calibration interval must be at least one breathing cycle (breath in and out). Thereby obtained signals are used to find the calibration curve by a numerical procedure. The calibration curve is than used to convert the curvature signal into the volume signal in further measurements.

In clinical applications, application of a single sensor is much simpler than the applications of a vest with sensors as proposed by PLPG method (Allsop T. et al., Application of long-period-grating sensors to respiratory Plethysmography, J Biomed Opt. 2007 November-December; 12(6):064003), or than application of a large number of sensors. This is of particular concern when dealing with critically ill patients, such as patients on mechanical ventilation. In addition, using large number of sensors may compromise the quality of measurements. The applicants have found that when the curvature sensor is placed on an area of the torso in which the underlying tissue is less stiff, like over abdomen or pectoralis muscle, the calibration curve becomes nonlinear. Thus, the application of a large number of sensors has a higher risk of yielding distorted results than the application of only one sensor.

The applicants have also found that the baseline drift inherent to the sensor signal or to the measurement method is not present in the measurements of curvature, meaning that the signal level corresponding to the FRC (Functional Residual Capacity—the volume of air present in the lungs at the end of expiration) is maintained for long periods of normal breathing with different tidal volumes. This implies that the measurement of the change in lung volume by curvature sensors may be successfully used for monitoring EELV (end-expiratory lung volume) with the FRC as a reference, at different levels of (PEEP—positive end-expiratory pressure) applied in mechanical ventilation. Methods that suffer from volume baseline drift, like RIP (Neumann P, Evaluation of Respiratory Inductive Plethysmography in Controlled Ventilation, Chest 1998; 113; 443-451) or PLPG (Allsop T. et al., Application of long-period-grating sensors to respiratory Plethysmography, J Biomed Opt. 2007 November-December; 12(6):064003) cannot be used for this purpose.

An additional advantage of using a single curvature sensor for monitoring respiration movements is the possibility to use its output as an input variable for triggering in mechanical ventilation and similar forms of breathing support. The change of the torso curvature happens shortly after the neural activity and the corresponding contraction of the main breathing muscle—diaphragm, with only 20 ms of the delay (Verbrugghe W, Jorens P G: Neurally adjusted ventilatory assist: a ventilation tool or a ventilation toy?, Respir Care. 2011 March; 56(3):327-35). The measurement of curvature of the torso surface provides a triggering variable that is independent of air leaks and the movements of the soft tissues that may provide bad signal-to-noise ratio. At the same time, this method for minimizing patient-ventilator asynchrony is noninvasive and thus simpler and more convenient for clinical application than the invasive synchronous neural triggering by a gastric catheter.

Since the main quality of the signal used for triggering is its phase synchronization with each breath and not the overall shape of the signal waveform, the quality of calibration in this case is less important than in the case of monitoring of the tidal volume. Moreover, the trigger may be realised by using an uncalibrated (raw) torso curvature signal only.

Another advantage of placing the sensor over a torso area with stiff underlying tissues is that this eliminates sharp bending that may lead to buckling of sensors observed in using a large number of sensors with some of them placed over the soft-tissue area (Allsop T. et al., Application of long-period-grating sensors to respiratory Plethysmography, J Biomed Opt. 2007 November-December; 12(6):064003). Sharp bending/buckling of sensors may cause multiple curvatures along the grating and thus lead to poor measurement results.

### BRIEF DESCRIPTION OF DRAWINGS

FIG. 1 is a schematic representation of a system for measuring respiratory volumes.

FIG. 2 is a schematic representation of (a) a long-period grating (LPG) curvature sensor and (b) an LPG curvature sensor encapsulated in silicone rubber.

FIG. 3 represents an LPG sensor transmission as a function of curvature.

FIG. 4 is a schematic representation of the interrogation module that is a part of the system shown in FIG. 1.

FIG. 5 is a plot of typical spirometer and curvature-sensor signals simultaneously measured on a patient, with a significant spirometer baseline drift.

FIG. 6 is a plot of typical spirometer and curvature-sensor signals simultaneously measured on a patient, with a large natural baseline drift in end-expiratory volumes.

FIG. 7 is a scatter plot of the signal obtained during the calibration process, Vs,corr(t)—the corrected spirometer signal, and Vc(t)—the calibrated curvature-sensor signal.

FIG. 8 shows an example of the calculated volumes compared to the referent spirometer signal.

FIG. 9 is a schematic representation of a system for triggering the breath initiation as a part of a mechanical ventilator.

FIG. 10 is a schematic representation of a system in which the optoelectronic module for breathing initiation detection and ventilator triggering uses electrocardiographic signals for reduction of a noise generated by mechanical pulsation of the heart.

### DESCRIPTION OF EMBODIMENTS

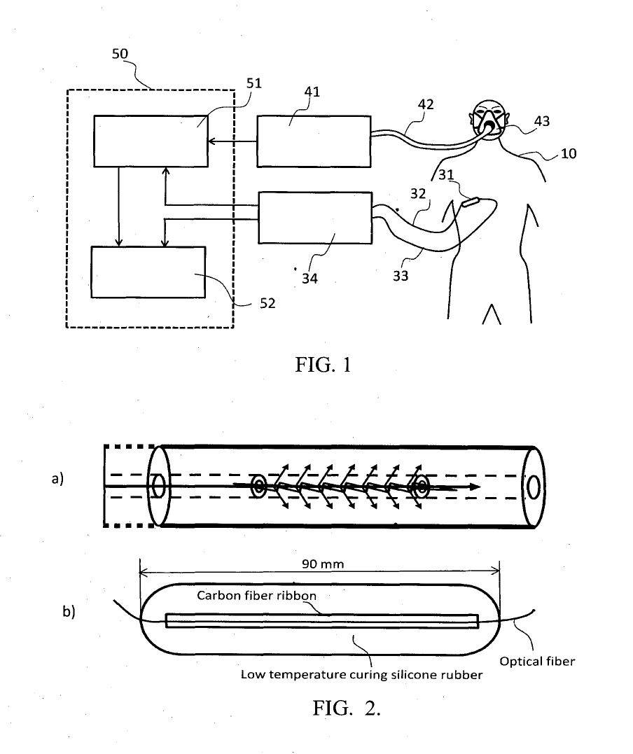

FIG. 1. shows a block diagram of an embodiment of a system for measuring respiratory volumes in accordance with the presented invention. Referring to FIG. 1, as an example of the sensor working principle, an LPG curvature sensor 31 is attached the patient's 10 lower ribs area, preferably between the ribs 6 and 8, between the lines parallel with the sternum, at about 10 cm left and right from the sternum. The sensor may be self-adhesive, placed on an adhesive tape, or attached otherwise. Two optical fibers 32 and 33 are connecting the LPG sensor to an interrogation module 34, which converts the optical signal from the LPG sensor 31 to a digital signal proportional to the sensor curvature. Since the measurements using LPG sensors are based on the transmitted signal detection, the direction of the optical signal propagation in the first optical fiber 32 is from the interrogation module 34 to the LPG curvature sensor 31, while in the other optical fiber 33 the transmitted optical signal travels in the opposite direction, from the LPG curvature sensor 31 to the interrogation module 34. An oronasal mask 43 is attached to the face of the patient 10 and connected to the pneumotachometer module 41 by a flexible tube 42. During the calibration phase of the measurement, the breathing volume signal from the pneumotachometer module 41 and the signal from the interrogation module 34 are sent to the calibration module 51. The calibration module 51 calculates the calibration function parameters by comparing these two signals. In the data acquisition phase, the acquisition module 52 acquires the calibration function parameters from the calibration module 51 and the curvature signal from the interrogation module 34, and calculates the respiratory volume. In the present embodiment, the calibration module 51 and the acquisition module 52 are parts of a programmable CPU 50 (CPU—Central Processing Unit). In another embodiment, comprising the system presented in FIG. 1 as an integral part of a mechanical ventilator, the calibration module 51 and the acquisition module 52 may be parts of the programmable CPU module of the mechanical ventilator.

If the present invention is used as a stand-alone device, then the oronasal mask 43 and the tube 42 should be placed on the patient 10 only during the calibration procedure. This also stands in the case when the present invention is used as a part of a diagnostic system which does not include an oronasal mask, as in the case of the laboratory-based polysomnography (PSG) used for obstructive sleep apnea syndrome (OSAS) diagnostics. Instead of an oronasal mask, a mouthpiece tube with a noseclip can be used.

If the present invention is used as a part of a therapeutic system that continuously uses an oronasal mask, such as a mechanical ventilator, the mask and the pneumotachometer that are parts of the mechanical ventilator can be used for providing reference breathing volume signal to the calibration module 51.

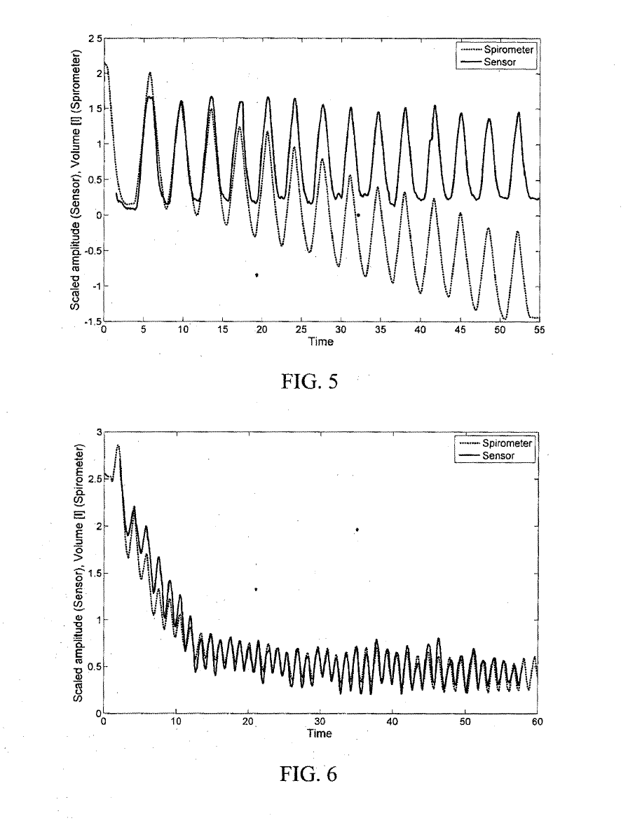

In the present embodiment the device used for reference respiratory volume measurement is a pneumotachometer. This type of device is most often used for volume measurement in mechanical ventilators. Pneumotachometers (or pneumotachographs) measure the flow according to the Venturi principle. The respiratory volume is then obtained by integrating the flow signal. Other types of respiratory-flow measuring devices such as ones based on turbine, ultrasound or hot wire anemometer, may also be used. The device used in the experimental measurements described by FIGS. 5,6,7 and 8 is an ultrasound spirometer.

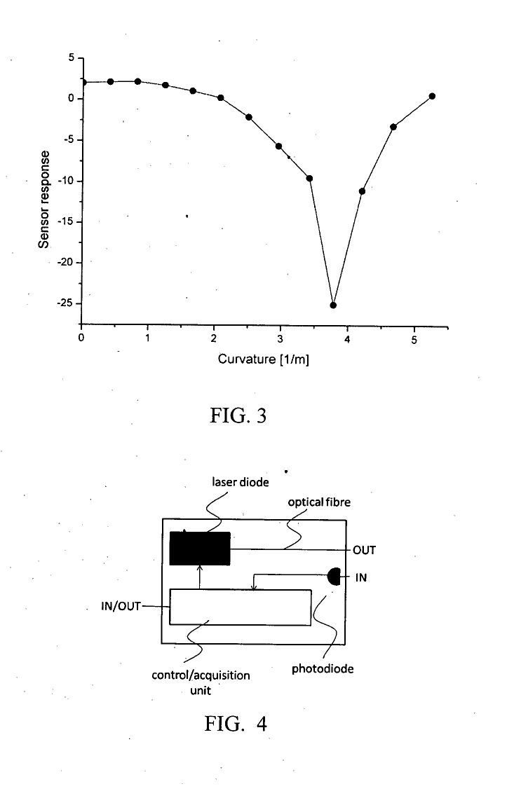

FIGS. 2a and 2b are schematic representations of a long-period grating (LPG) curvature sensor. The LPG consists of a periodic change in the refractive index or the fiber geometry along the fiber, with a typical period of several hundred micrometers. It couples the light from the core mode to the resonant co-propagational cladding modes of the fiber. These cladding modes are being absorbed by the coating which results in appearance of attenuation bands in the transmission spectrum. Both the resonant wavelength and the magnitude of an attenuation band are sensitive to forces applied to the fiber (strain, bending) and environmental conditions (temperature, external refractive index). FIG. 3 shows an example of the transmission dependence on the curvature of an LPG sensor used in the breathing volume measurement. The sensor sensitivity to the external refractive index can be eliminated by encapsulating the sensor into some elastic material like silicone rubber. The sensitivity to the temperature is not an issue for the sensor application on the human torso due to the small temperature variations of the human body.

In the present embodiment, the LPG curvature sensor is made as shown in FIG. 2b. The LPG is encapsulated in a silicone rubber, for the purposes of mechanical protection and reduction of the sensor cross-sensitivity to temperature and external refractive index. Other realisations of LPG curvature sensors may also be used, like the one with two rubber layers (Allsop T. et al., Embedded progressive-three-layered fiber long-period gratings for respiratory monitoring. J Biomed Opt. 2003 July; 8(3):552-8), without changing the essence of the present invention.

FIG. 3. Is a representation of the transmission of an LPG sensor as a function of curvature.

In another embodiment of the present invention, the curvature sensor could be based on a fiber Bragg grating (FBG). A fiber Bragg grating (FBG) is a type of distributed Bragg reflector inscribed in a short segment of an optical fiber that reflects light at particular wavelengths, while transmitting all the others. An advantage of using FBG sensors is that they are less sensitive to the parameters of the environment. However, the LPG sensors are more sensitive to the curvature changes than the FBG sensors since the resonant cladding modes of the LPG sensor sense a bend-induced refractive index change across the whole cross section of the fiber, while the back-propagating core modes generated by an FBG sense only a change in the refractive index of the core.

In another embodiment of the present invention, a curvature sensor based on a resistive strain gauge or a similar device for curvature measurement may be used.

FIG. 4 is a schematic representation of the interrogation module 34. The scheme of the interrogation module 34 used in the present embodiment is based on measuring the light power at the output of the sensor. The interrogation module consists of a fiber-coupled narrowband laser with control and stabilization units, for instance a temperature and current stabilized laser diode, and a photodiode that converts the optical signal from the output of the sensor into the electrical signal available at the output of the module. This scheme can be replaced by an equivalent scheme that allows for the light-power measurement at a single wavelength.

### Calibration Procedure

During the calibration procedure, the breathing volume signal from the pneumotachometer module 41 and the interrogation module 34 are sent to the calibration module 51 (FIG. 1). A short period of breathing (at least one full breath—inspiration and expiration) is needed for calibration purposes.

FIG. 5 is a plot of a typical case of simultaneous measurements by a spirometer and a curvature sensor on a patient, whereby a significant spirometer baseline drift can be observed. It can be seen that, unlike spirometer, curvature sensor signal has a steady base line. The spirometer used for this measurement was SpiroTube, Thor Medical, Budapest, based on ultrasound measurement of air velocity in two directions—during inspiration and expiration. The volumes in spirometer measurement are obtained by integration of air flows, which are obtained from the instantaneous air velocities. The integration causes a baseline drift. These drifts are intrinsic to the measuring method, since the speed of sound depends on the temperature, humidity and pressure of the flowing air, which may be different during inspiration and expiration. Although these dependences are compensated in different ways in more sophisticated types of pneumotachometers, baseline drifts remain inevitable in flow-based measurements of respiratory volumes. Even in the most sophisticated devices, such as D-lite flow and airway pressure sensor (GE Healthcare, Helsinki, Finland), tidal volume errors are reported to be within a range of +/−5% (Grivans C. et al., Positive end-expiratory pressure-induced changes in end-expiratory lung volume measured by spirometry and electric impedance tomography Acta Anaesthesiol Scand 2011; 55: 1068-1077).

FIG. 6 is a plot of a typical case of simultaneous measurements by spirometer and curvature sensor on a patient during which a large natural drift in end-expiratory volumes is observed. This example shows that a patient may change the level of the end-expiratory volume for about 2 liters during one minute breathing while maintaining roughly constant tidal volumes of around 0.5 liters.

The calibration procedure used in the present embodiment is based on the assumption that the baseline drift in the spirometer or pneumotachometer volume measurements is a sum of the baseline drifts due to a) the systematic volume measurement error and b) the natural change in end-expiratory volumes, while the baseline drift in the curvature measurement is caused only by the natural change in end-expiratory volumes. The assumption is made upon the observation that the base level of the curvature sensor signal remains constant when the end-expiratory volume is maintained at a fixed value over a long curvature/respiration measurement.

The baseline drift due to the measurement error in the spirometer/pneumotachometer signal Vs(t) is described by the function Ds(t), and the baseline change of the curvature measurement Vc(t) corresponding to the natural change in end-expiratory volumes is described with the function Dn(t). In the present embodiment, functions Ds(t) and Dn(t) are obtained by the 2^{nd} order polynomial interpolation of the 1 min calibration signals Vs(t) and Vc(t), respectively. In other embodiments, functions Ds(t) and Dn(t) can be obtained by using different procedures, such as finding the minimums of signals Vs(t) and Vc(t), whereby these minimums correspond to the end-expiratory volumes, and then performing a polynomial interpolation of these minimum points, by using a digital low-pass filter, etc.

The spirometer/pneumotachometer signal corrected for the measurement error caused by the baseline drift Vs,corr(t) is then obtained by subtracting the difference of functions Ds(t) and Dn(t) from the original spirometer signal Vs(t):

Vs,corr(t)=Vs(t)−(Ds(t)−Dn(t))

In this way, the calibration procedure used in the present embodiment eliminates the excess baseline drift in the reference volume measurement (example of which is shown in FIG. 5) without distorting the natural change in end-expiratory volumes (example of which is shown in FIG. 6.).

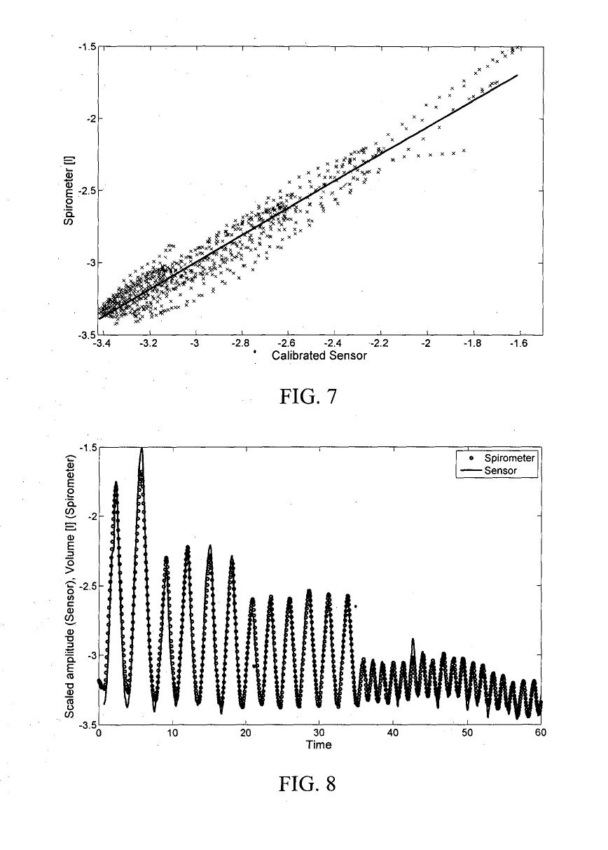

After the baseline drift correction, two arrays of signal points are obtained for a calibration period: Vs,corr(t)—the spirometer/pneumotachometer signal, and Vc(t)—the curvature signal. These two signals are depicted in a scatter plot in FIG. 7.

In the present embodiment, the curvature Vc(t)—volume Vt(t) calibration function is a linear function

Vt(t)=K1+K2Vc(t),

with the constants K1 and K2 obtained by applying the least squares regression to Vs,corr(t) and Vc(t):

$$K 2 = \frac{{\int }_{0}^{T} Vs , corr ( t ) * Vc ( t ) d t - \frac{1}{T} {\int }_{0}^{T} Vs , corr ( t ) d t * {\int }_{0}^{T} Vc ( t ) d t}{{\int }_{0}^{T} {[ Vc ( t ) ]}^{2} d t - \frac{1}{T} {( {\int }_{0}^{T} Vc ( t ) d t )}^{2}}$$

$$K 1 = \frac{1}{T} ( {\int }_{0}^{T} Vs , corr ( t ) d t - K 2 {\int }_{0}^{T} Vc ( t ) d t ) ,$$

where T is the measurement time interval.

In other embodiments, this function may be a higher order order polynomial.

The constants K1 and K2 obtained from a simultaneous calibration measurement of the curvature and spirometer/pneumotachometer signals are then used in the calculation of respiratory volumes in the subsequent curvature measurements. An example of thus measured and calculated volumes compared to the referent spirometer/pneumotachometer signals for three different tidal volumes during a one-minute measurement, is shown in FIG. 8.

The embodiment shown in FIG. 1 may also be used for diagnosing sleep related breathing disorders, such as Obstructive Sleep Apnea. (OSA), as a part of Polysomnographic devices. It can also be used in devices for the treatment of similar disorders, like Continuous Positive Airway Pressure (CPAP) and Bilevel Positive Airway Pressure (BPAP) devices. In these devices, the curvature-based volume measurement may be used to monitor therapy efficiency, as well as for pressure titration—a method for choosing optimal therapeutic pressure in such devices.

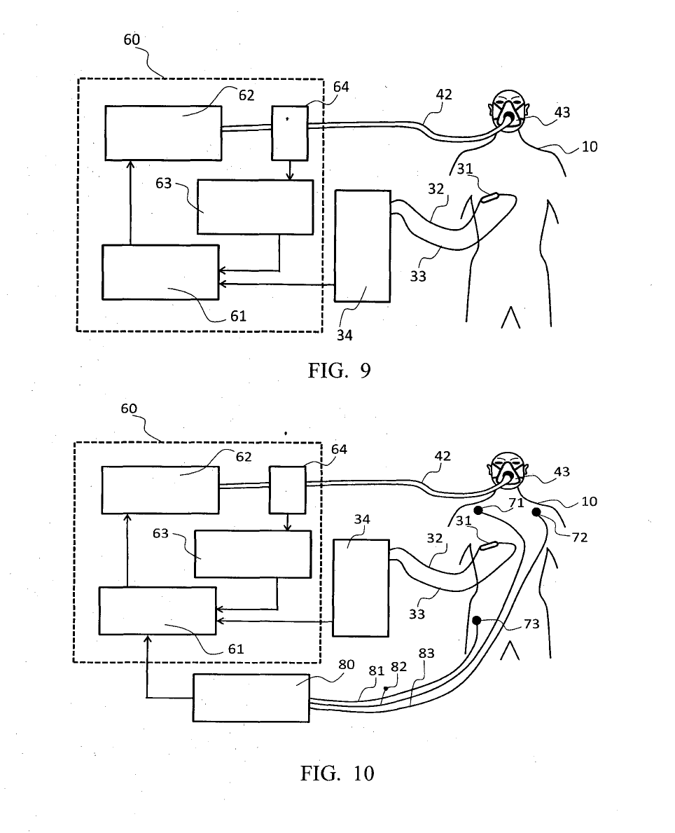

FIG. 9 is a schematic block diagram of an embodiment of a system for triggering the breath initiation as a part of a mechanical ventilator in accordance with the present invention. When the curvature measurement is used only for triggering, and not for monitoring of respiration volumes, then the calibration elements of the system are not needed and the corresponding device is simpler than that in the embodiment shown in FIG. 1. The curvature sensor 31, attached to the torso of the patient 10, is connected to the interrogation module 34 by optical fibers 32 and 33. The interrogation module 34 converts the optical signal from the LPG sensor 31 to a digital signal proportional to the sensor curvature. The CPU module 61 of the mechanical ventilator 60 then uses the curvature signal as a triggering signal for breath initiation by the air pump 62 of the mechanical ventilator 60. In another embodiment, the CPU module 61 may also use a combination of the curvature signal and other pneumatic signals (flow, pressure, volume) from a pneumatic sensor 63 and its pick-up 64 on the flexible tube 42 for breath initiation by the air pump 62.

The embodiment depicted in FIG. 9 may also be used for triggering of different phases of mechanical ventilation cycle other than breath initiation, such as initiation of expiration, etc.

In the embodiment in which the described device is used as a part of a mechanical ventilator for triggering of the breath initiation, the signal to noise ratio is very important, particularly in the phase of the breath initiation, the most important phase for the triggering purposes and at the same time a phase in which change of the breathing curvature (signal) is small. The mechanical pulsation of the human heart produces movements of the torso surface that may be comparable in magnitude with the breathing movements. The signal from the heart pulsations is larger when the curvature sensor is attached to the left side of the torso, near the heart apex, but it is always present on the whole torso surface and hence may produce a significant noise in the signal of the breathing movement. This noise may be eliminated by the method described in the present invention. The method is based on the fact that a) electrocardiogram (ECG) signal of a particular heart beat starts earlier than the mechanical heart pulsation; b) ECG and heart pulsation signals of an individual have very repeatable waveforms, and c) the time interval between these two signals is practically constant for a constant heart rate (Weissler A. et al., Systolic Time Intervals in Heart Failure in Man, Circulation 1968; 37; 149-159); the time interval between the start of the electrical depolarization of the heart (the Q point of the QRS complex in ECG signal) and the start of the heart pulsation signal being about 100 ms for a healthy individual (Weissler A. et al., Systolic Time Intervals in Heart Failure in Man, Circulation 1968; 37; 149-159).

FIG. 10 shows a schematic representation of a device in which the module used for the detection of breath initiation and the ventilator activation (triggering), uses ECG signals for the elimination of the noise caused by mechanical heart pulsation. In this embodiment, the ECG acquisition module 80 is connected to the patient 10 by cables 81, 82 and 83 and electrodes 71, 72 and 73. The ECG module 80 converts the voltages from the electrodes attached to the body surface to a digital ECG signal. The ECG module 80 is connected to the CPU module 61 of the mechanical ventilator 60, so that the digital ECG signal is continuously sent to the CPU module 61. The CPU module uses the ECG signal to eliminate the heart pulsation signal from the curvature sensor in the following manner:
1. The calibration phase comprises a short period of about 10 heart beats, but at least one complete heart beat, during which the curvature and ECG signals are measured simultaneously and stored in the CPU module 61. During this measurement, the patient is asked not to breath. If this is not possible, a high-pass digital filter is used to eliminate the component of the signal which is due to breathing.
2. A representative heart beat is selected during the calibration according to the quality of the ECG and curvature signals in that heart beat.
3. A reference point is detected in the representative heart beat in the ECG signal. In the present embodiment the Q point (the starting point of the QRS complex in ECG signal) is used as a reference.
4. A starting point S is detected in the representative heart beat in the curvature signal.
5. The time delay Tqs between the Q point of the ECG signal and the S point of the curvature signal is calculated using the selected representative heart beat.
6. During the continuous operation after the calibration phase, the Q point of each QRS complex is detected in the ECG signal, and the representative heart beat signal obtained from the curvature sensor in the calibration phase is delayed for Tqs with respect to the corresponding Q point and subtracted from the actual signal of the curvature sensor.

The representative-beat selection and the detection of the Q and S points can be done manually or by using software tools. Also, a median beat calculated from some time interval may be used as a representative beat.

A characteristic point of the ECG signal other than the Q point, for instance P point (the starting point of the P wave), can be used as a reference point. Also, a point other then the starting point S of the heart beat waveform can be used as a reference point in the curvature signal.

The present embodiment uses three ECG electrodes for measuring ECG signal: two active measuring electrodes and one ground electrode. Other configurations for measuring one or more ECG signals can also be used.

In the embodiments shown in FIG. 9 and FIG. 10, the present invention is used as an integral part of a mechanical ventilator, and uses the CPU of the said ventilator. In other embodiments, the present invention may be a stand-alone device that would send a triggering signal to a mechanical ventilator. In such embodiments, the stand-alone device would comprise a programmable CPU module that would be used for similar purposes as the CPU module 61 shown in FIG. 9 and FIG. 10.

1-22. (canceled)

23. A device for determining respiratory-induced movement, comprising:
one curvature sensor attachable to a highly-stiff area of a patient's torso and operable to generate a signal that is a function of a curvature change in said area; and
a processor operable correlate the signal from only the one curvature sensor to respiratory-induced movement of the patient and to generate an output signal indicative of said respiratory-induced movement.

24. The device of claim 23, wherein the processor is further operable to determine change in lung volume using the generated output.

25. The device of claim 24, wherein the processor determines at least one of tidal volume or a change in end-expiratory lung volume (EELV) based on the change in lung volume.

26. The device of claim of claim 23, wherein the generated output signal is operable to trigger a ventilator.

27. The device of claim 23, wherein the processor is further operable to generate a ventilator triggering signal by filtering out heart pulsation signals from said generated output and to trigger a ventilator using the ventilator triggering signal.

28. The device of claim 27, wherein the processor is operable to perform the filtering by subtracting a representative heart pulsation signal from said generated output.

29. The device of claim 28, wherein the processor is operable to synchronize to an ECG said subtracted representative heart pulsation signal.

30. The device of claim 23, wherein the curvature sensor is a long period grating (LPG) sensor in an optical fiber, a fiber Bragg grating (FBG) sensor in an optical fiber, or a strain gauge.

31. The device of claim 23, wherein the processor is further operable to:
calibrate the generated output to compensate reference volume measurement baseline drift by:
obtain a reference volume measurement Vs(t);
determine a baseline drift Ds(t) reference volume measurement Vs(t);
determine baseline drift Dn(t) due to natural change in end-expiratory volume; and
subtract a difference Ds(t)−Dn(t) from the volume measurement Vs(t).

32. The device of claim 23, further comprising:
a fiber-coupled narrowband laser with stabilization and control units; and
a photodiode for conversion of an optical signal from the curvature sensor into electrical signal.

33. A method for detecting respiratory-induced movement, comprising:
placing one curvature sensor at a highly-stiff area of a patient's torso;
detecting curvature change signals from the curvature sensor; and
generating an output indicative of respiratory-induced movement based on the detected curvature change signals from only the one curvature sensor.

34. The method of claim 33, further comprising determining change in lung volume using the generated output.

35. The method of claim 34, further comprising determining at least one of tidal volume or a change in end-expiratory lung volume (EELV) based on the change in lung volume.

36. The method of claim of claim 33, further comprising triggering a ventilator using the generated output.

37. The method of claim 33, further comprising generating a ventilator triggering signal by filtering out heart pulsation signals from said generated output, and triggering a ventilator using the ventilator triggering signal.

38. The method of claim 37, wherein the filtering is performed by subtracting a representative heart pulsation from said generated output.

39. The method of claim 38, wherein said subtracting a representative heart pulsation signal is synchronized to an ECG signal.

40. The method of claim 33, wherein the curvature sensor is a long period grating (LPG) sensor in an optical fiber, a fiber Bragg grating (FBG) sensor in an optical fiber, or a strain gauge.

41. The method of claim 33, further comprising:
calibrating the generated output to compensate reference volume measurement baseline drift by:
obtaining a reference volume measurement Vs(t);
determining a baseline drift Ds(t) reference volume measurement Vs(t);
determining baseline drift Dn(t) due to natural change in end-expiratory volume; and
subtracting a difference Ds(t)−Dn(t) from the volume measurement Vs(t).

42. The method of claim 41, wherein the reference volume measurement is obtained by a pneumotachometer or a spirometer.

43. The method of claim 33, wherein the highly-stiff area is between the ribs 6 and 8.
